(19) United States
(12) Patent Application Publication
(10) Pub. No.: US 2008/0051427 A1
(43) Pub. Date: Feb. 28, 2008

(54) Pharmaceutical Compositions and Methods of Using Same

(76) Inventor: Fritz Schuckler, Bergisch-Gladbach (DE)

Correspondence Address: GOODWIN PROCTER LLP;PATENT ADMINISTRATOR, EXCHANGE PLACE, BOSTON, MA, 02109-2881, US

(21) Appl. No.: 11/750,556

(22) Filed: May 18, 2007

(30) Foreign Application Priority Data
May 18, 2006 (EP) 06010232.4

(51) Int. Cl. A61K 31/437; A61P 3/06

(52) U.S. Cl. 514/292

(57) ABSTRACT

A pharmaceutical composition is provided that comprises a solid dispersion of implitapide. Such solid dispersions may include implitapide and least one pharmaceutically acceptable excipient. In some embodiments, the disclosed solid dispersions comprise substantially amorphous implitapide.

### RELATED APPLICATIONS

This application claims priority to European Patent Application EP06010232.4, filed May 18, 2006, which is hereby incorporated by reference in its entirety.

### FIELD

This invention relates to pharmaceutical compositions and their use in, for example, treating hyperlipidemic disorders. The disclosed pharmaceutical compositions can be used as a sole agent or in combination with other therapies.

### BACKGROUND

Hypercholesterolemia and hyperlipidemia are considered major risk factors fro the development of coronary heart disease. Various epidemiological studies have demonstrated that drug mediated lowering of total cholesterol and low density lipoprotein (LDL) cholesterol is associated with a significant reduction in cardiovascular events. The National Cholesterol Education Program's (NCEP's) updated guidelines recommends that the overall goal for high-risk patients is to achieve less than 100 mg/dL of LDL, with a therapeutic option to set the goal for such patients to achieve a LDL level less than 70 mg/dL.

Microsomal triglyceride transfer protein (MTP) inhibitors have been developed as potent inhibitors of MTP-mediated neutral lipid transfer activity. Microsomal triglyceride transfer protein (MTP) is essential for the synthesis of both chylomicron in the intestine and very low-density lipoprotein in the liver. MTP is a heterodimeric transfer protein which also limits the production of atherogenic apolipoprotein B (apoB)-containing lipoproteins. MTP, therefore, is one target for the treatment of, e.g. dyslipidemias and treatment and/or prevention of atherosclerosis.

Implitapide is one such compound that has been shown to inhibit apoB-lipoprotein secretion from liver cells and diasteroselectively inhibit MTP-catalyzed transport of lipids.

### SUMMARY

A pharmaceutical composition is provided that comprises a solid dispersion. Such a solid dispersion includes implitapide and a pharmaceutically acceptable matrix. Contemplated compositions and/or solid dispersions include those with a weight ratio of implitapide to a pharmaceutically acceptable matrix of about 1:3 to about 1:9, for example, about 1:3 to about 1:4.

In some embodiments, a substantial portion of the implitapide in the disclosed compositions is in an amorphous state. Such compositions may include those where the solubility of implitapide is increased as compared to the solubility of crystalline implitapide, for example, the solubility is increased by at least 400-fold over that of crystalline implitapide.

The compositions include a pharmaceutically acceptable matrix that can, for example, comprise at least one of: a sugar, cyclodextrin, or a sugar alcohol. In a different embodiment, the pharmaceutically acceptable matrix can comprise a pharmaceutically acceptable polymer, for example, polyvinylpyrrolidone and/or hydroxypropylcellulose. Disclosed compositions can include additional active ingredients, e.g. those useful for the treatment of hyperlipidemic diseases.

The disclosed compositions can be suitable for oral administration, e.g. can be in the form of a tablet, for example, an immediate release tablet.

In an embodiment, a composition may comprise a solid dispersion comprising implitapide, wherein said composition, when administered to a patient, results in a higher exposure, as measured by AUC, of implitapide, as compared to administering to a patient a suspension of substantially crystalline implitapide. The higher exposure may be at least about 7-fold higher, at least about 10-fold higher, about 10-fold to about 20-fold higher, or even at least about 20-fold higher.

In another embodiment, a composition comprising implitapide and a pharmaceutically acceptable matrix is provided, wherein the weight ratio of the implitapide to the pharmaceutically acceptable matrix is about 1:3 to about 1:4.

Processes for manufacturing a disclosed pharmaceutical composition are also disclosed herein. Such processes may include: a) dissolving the implitapide and at least one pharmaceutically acceptable matrix in solvent or a solvent mixture, e.g. a solvent or solvent mixture that includes acetone, to form a solution; b) contacting the solution with one or more pharmaceutically acceptable excipients; c) removing said solvent or solvent mixture to form a granulate; and d) optionally blending said granulate with one or more further pharmaceutically acceptable excipients to form post-blend granulates. Disclosed processes may further comprise subdividing said post-blend granulates, and optionally further comprise coating said post-blend granulates with one or more further pharmaceutically acceptable excipients.

In an embodiment, a method for treating a hyperlipidemic disorder in a patient in need thereof is provided, comprising administrating a pharmaceutically effective amount of a disclosed pharmaceutical composition.

### BRIEF DESCRIPTION OF FIGURE

FIG. 1 depicts a comparison of milled crystalline drug in a suspension and a coprecipitate tablet (n=6 animals) in a dog kinetic study.

### DESCRIPTION

This disclosure is directed, at least in part, to the use of a pharmaceutical composition comprising a solid dispersion of the compound of Formula I. Such compositions may provide significant advantages for a patient.

Formula I can be depicted as:

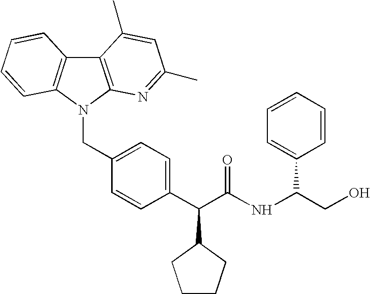

The term “implitapide,” “the compound of Formula I,” or “the compound of this invention” refers to (2S)-2-cyclopentyl-2-(4-((2,4-dimethyl-9H-pyrido(2,3-B)indol-9-yl)methyl)-phenyl)-N-((1R)-2-hydroxy-1-phenylethyl)acetamide, as depicted in Formula I, and in certain embodiments, also refers to its polymorphs, solvates, hydrates, pharmaceutically acceptable salts, or a combination thereof.

The present invention pertains to, at least in part, pharmaceutical compositions containing the compound of Formula I in the form of a solid dispersion (i.e. formulations rendering the drug substance from a predominantly crystalline status into a predominantly to perfect amorphous status), which includes e.g. solid solutions, glass solutions, glass suspensions, amorphous precipitations in a crystalline carrier, eutectics or monotectics, compound or complex formation and combinations thereof.

Also contemplated herein is the use of the disclosed compositions for the treatment of hyperlipidemic diseases, either as a sole agent, or in combination with other lipid lowering therapies.

Disclosed herein is a formulation or composition that includes a solid dispersion of implitapide. For example, such formulation may include one part of the compound and e.g. about 3 to about 9 parts of a matrix forming agent, e.g. a pharmaceutically acceptable matrix. Formulations may include a weight ratio of implitapide to pharmaceutically acceptable matrix of about 1:3, 1:4, 1:5, 1:6; 1:7; 1.8, 1:9 or even about 1:10. In an embodiment, about 3 to about 4 parts of the matrix forming agent may be present in such a composition to about one part implitapide. Such compound/matrix forming agent ratios are capable of increasing the solubility of this drug substance up to multiple hundred-fold, e.g. at least 100-, 200-, or even 400-fold. Such formulations can also lead to a tremendous increase of the efficacy in-vivo of the compound, compared with the compound in the crystalline state, and thus provide a solid pharmaceutical dosage form with convenient size.

In the following, the different types of solid dispersions (e.g. solid solutions, glass solutions, glass suspensions, amorphous precipitations in a crystalline carrier, eutectics or monotectics, compound or complex formation and combinations thereof) are collectively referred to as solid dispersion.

In an aspect, a pharmaceutical composition disclosed herein comprises a solid dispersion comprising at least the compound of Formula I and a pharmaceutically acceptable matrix.

In one aspect, a pharmaceutical composition is provided that comprises a solid dispersion, wherein the matrix comprises a pharmaceutically acceptable polymer, such as polyvinylpyrrolidone, vinylpyrrolidone/vinylacetate copolymer, polyalkylene glycol (i.e. polyethylene glycol), hydroxyalkyl cellulose (i.e. hydroxypropyl cellulose), hydroxyalkyl methyl cellulose (i.e. hydroxypropyl methyl cellulose), carboxymethyl cellulose, sodium carboxymethyl cellulose, ethyl cellulose, polymethacrylates, polyvinyl alcohol, polyvinyl acetate, vinyl alcohol/vinyl acetate copolymer, polyglycolized glycerides, xanthan gum, carrageenan, chitosan, chitin, poyldextrin, dextrin, starch and proteins.

Another aspect provided herein is a pharmaceutical composition comprising a solid dispersion that includes a matrix, wherein the matrix comprises a sugar and/or sugar alcohol and/or cyclodextrin, for example sucrose, lactose, fructose, maltose, raffmose, sorbitol, lactitol, mannitol, maltitol, erythritol, inositol, trehalose, isomalt, inulin, maltodextrin, β-cyclodextrin, hydroxypropyl-β-cyclodextrin or sulfobutyl ether cyclodextrin.

Additional suitable carriers that are useful in the formation of the matrix, or may be included in a composition comprising a disclosed solid dispersion include, but are not limited to alcohols, organic acids, organic bases, amino acids, phospholipids, waxes, salts, fatty acid esters, polyoxyethylene sorbitan fatty acid esters, and urea.

A solid dispersion of the compound of Formula I in the matrix may contain certain additional pharmaceutical acceptable ingredients, such as surfactants, fillers, disintegrants, recrystallization inhibitors, plasticizers, defoamers, antioxidants, detackifier, pH-modifiers, glidants and lubricants.

The solid dispersion of the invention can be prepared according to methods known to the state of the art for the manufacture of solid dispersions, such as fusion/melt technology, hot melt extrusion, solvent evaporation (i.e. freeze drying, spray drying or layering of powders of granules), coprecipitation, supercritical fluid technology and electrostatic spinning method.

In one embodiment, a pharmaceutical composition is provided in which the compound of Formula I is substantially amorphous.

Another aspect disclosed herein is a solid dispersion of the compound of Formula I, wherein the matrix is a polyvinylpyrrolidone polymer.

Another aspect is a solid dispersion of the compound of Formula I, wherein the matrix is a hydroxypropylcellulose polymer.

The pharmaceutical composition provided herein can be utilized to achieve desired pharmacological effects by, e.g., oral administration to a patient in need thereof, and can be advantageous to a conventional formulation (e.g. with the drug in the crystalline state) in terms of drug release, bioavailability, and/or interpatient variability in mammals. A patient, for the purpose of this invention, is a mammal, including a human, in need of treatment for the particular condition or disease.

For oral administration, the solid dispersion described herein can be formulated into solid or liquid preparations such as powder, granules, pellets, tablets, capsules, dragees, chewable tablets, dispersible tables, troches, lozenges, melts, solutions, suspensions, or emulsions, and may be prepared according to methods known to the state of the art for the manufacture of pharmaceutical compositions. For this purpose the solid dispersion may be compounded with conventional excipients, for example binders, fillers, lubricants, disintegrants, solvents, surfactants, thickeners and stabilizers, coating materials as well as flavoring agents, sweeteners, flavoring and coloring agents.

It is believed that one skilled in the art, utilizing the preceding information, can utilize the present invention to its fullest extent. The oral formulation of the compound of Formula I refers to a wide range of dosages such as 1 mg, 10 mg, 100 mg, or even 1 g daily dosing and beyond. This would be accomplished, for example, by modifying the composition and size of the tablet or capsule, and/or by administering multiple tablets or capsules per day to the patient in need thereof. Alternatively, the solid dispersion formulation may also be dosed in forms such as powders, granules, chewable or dispersable tablets, or by dispersions of any adequate solid formulation in a suitable liquid prior to use, for example if the optimal dose regimen was no longer consistent with a feasible tablet or capsule size.

The total amount of the active ingredient (i.e. a compound of Formula I) to be administered via an oral route using the new pharmaceutical composition of the present invention will generally range from about 0.01 mg/kg to about 10 mg/kg body weight per day. A unit dosage may contain from about 1 mg to about 500 mg of active ingredient, preferably from 5 mg to 100 mg of active ingredient, e.g. about 5 mg, 10 mg, 15 mg, 20 mg, 30 mg, 40 mg, 50 mg, 80 mg or 100 mg, and can be administered one or more times per day, typically one, two or three times a day.

The pharmaceutical compositions of this invention can be administered as the sole agent or in combination with one or more other therapies where the combination causes no unacceptable adverse effects.

It is believed that one skilled in the art, using the preceding information and information available in the art, can utilize the present invention to its fullest extent. It should be apparent to one of ordinary skill in the art that changes and modifications can be made to this invention without departing from the spirit or scope of the invention as it is set forth herein.

Examples 1, 2 and 3 refer to different preparations of solid dispersions of the compound of this invention (powder and tablet). In vivo testing of representative solid dispersion formulations of the compound of this invention are described in Examples 4 (in dogs) and 5 (in humans).

### EXAMPLES

### Example 1

Preparation of 1:3, 1:4, 1:6, and 1:9 solid dispersion of the compound of Formula I with polyvinylpyrrolidone

In an uncapped vial, one part of the compound of Formula I is mixed with three, four, six, or nine parts polyvinylpyrrolidone (PVP-25/Kollidon 25), respectively. The mixture is dissolved in a sufficient amount of a mixture of acetone and ethanol, until all powders were in solution. The uncapped vial is placed into a vacuum oven set at 40° C., and let dry for at least 24 hours.

After that treatment, an amorphous state of the powder is achieved, which could be identified, e.g. by X-ray diffraction measurements or by determination of the solubility. For example, a 1:3 ratio of drug to PVP result in approximately a 400-fold increase in solubility (40 mg/l vs. 0.1 mg/l in 0.1 N HCl).

### Example 2

Manufacture of a tablet formulation based on a solid dispersion of the compound of Formula I with polyvinylpyrrolidone ratio 1:4

The drug of Formula I, together with polyvinylpyrrolidone (ratio 1:4) is added to a mixture of acetone and ethanol (ratio drug:acetone:ethanol=1:24:6.4) and stirred until a clear solution is achieved (warming up of the batch if necessary). This solution is consequently sprayed onto a powder base, containing microcrystalline cellulose and croscarmellose sodium, in a fluidized granulation process, resulting in a granulate containing the drug in the state of a coprecipitate. In order to reduce a high bulk volume, the granulate is treated by roller compaction. In the next step, the post blend components croscarmellose sodium and magnesium stearate are added. After blending, tablets are compressed on a suitable tabletting machine, and finally the tablets are film-coated (standard coating layer based on Hypromellose). A typical composition is the following (for example, tablets 5 mg and tablets 20 mg):

| Component [mg/tablet] | Tablets 5 mg | Tablets 20 mg |
|---|---|---|
| Compound of formula 1 | 5.000 | 20.000 |
| Polyvidone 25 | 20.000 | 80.000 |
| Croscarmellose sodium | 34.300 | 137.200 |
| (powder base + postblend) | (23.100 + 11.200) | (92.400 + 44.800) |
| Microcrystalline cellulose | 23.100 | 92.400 |
| Magnesium stearate | 0.200 | 0.800 |
| uncoated tablet | 82.600 | 330.400 |
| Hypromellose 15 cP | 1.440 | 5.760 |
| Macrogol 4000 | 0.480 | 1.920 |
| Yellow ferric oxide | 0.096 | 0.384 |
| Titanium dioxide | 0.384 | 1.536 |
| coated tablet | 85.000 | 340.000 |
| Tablet size | round; | oblong |
| | diameter 6 mm | 14 mm length × 6 mm |
| | | width |

### Example 3

Manufacture of a tablet formulation based on a solid dispersion of the compound of Formula I with polyvinlylpyrrolidone ratio 1:3

The drug of formula I, together with polyvinylpyrrolidone (ratio 1:3) is added to a mixture of acetone and ethanol (ratio drug:acetone:ethanol=1:24:6.4) and stirred until a clear solution is achieved. To facilitate this step, the batch is slightly warmed up. This solution is consequently sprayed onto a powder base, composed of croscarmellose sodium, in a fluidized granulation process, resulting in the state of a coprecipitate. In order to reduce a high bulk volume, the granulate is treated by roller compaction. In the next step, the post blend components croscarmellose sodium and magnesium stearate are added. After blending, tablets are compressed on a suitable tabletting machine, and finally the tablets are film-coated (standard coating layer based on Hypromellose).

A typical composition is the following (for example, tablets 40 mg and 80 mg):

| Component [mg/tablet] | Tablets 40 mg | Tablets 80 mg |
|---|---|---|
| Compound of formula 1 | 40.000 | 80.000 |
| Polyvidone 25 | 120.000 | 240.000 |
| Croscarmellose sodium | 182.400 | 364.800 |
| (powder base + postblend) | (106.700 + 75.700) | (213.400 + 151.400) |
| Magnesium stearate | 1.700 | 3.400 |
| uncoated tablet | 344.100 | 688.200 |
| Hypromellose 15 cP | 4.740 | 7.080 |
| Macrogol 4000 | 1.580 | 2.360 |
| Yellow ferric oxide | 0.320 | 0.482 |
| Titanium dioxide | 1.260 | 1.888 |
| coated tablet | 352.000 | 700.000 |
| Tablet size | oblong; | oblong; |
| | 14 mm length × | 16 mm length × 8 mm |
| | 6 mm width | width |

### Example 4

In-vivo testing/dog kinetic trials of different formulations containing the compound of Formula I

In a kinetic study in dogs, the compound of Formula I is administered to the animals in several formulation principles at a dose of 1 mg/kg body weight:
solid dispersion according to example 1 (ratio 1:3)
suspension with crystalline, micronized drug substance
“nanosuspension” with crystalline drug substance with a mean particle size d₅₀<1 μm

The absorption of the crystalline drug is found to be so poor that no plasma levels could be detected; only the solid dispersion led to reasonable plasma levels and calculable PK parameters.

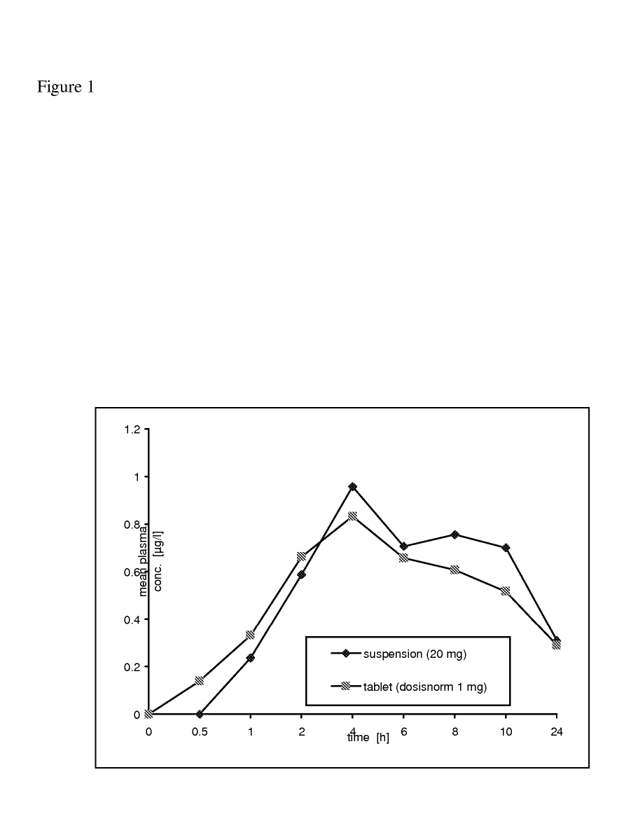

When comparing a tablet formulation of the compound of formula I according to example 2 (ratio 1:4) with a suspension of the crystalline compound (milled down to provide a “nanosuspension” with a mean particle size d₅₀<1 μm) in a dog kinetic trial, it can be found that the formulation according to example 1 resulted in approximately 20-fold higher exposure with respect to AUC values (see FIG. 1).

### Example 5

In-vivo testing/human trial of different formulations containing the compound of Formula I

When comparing the tablet formulation of the compound of Formula I according to example 2 (dose strength=1 mg) with a crystalline suspension of this compound (dose=20 mg; milled down to a “nanosuspension” with a mean particle size d₅₀<1 μm) in a standard Phase I human trial, it can be found that the formulation according to example 1 results in approximately 7-fold higher exposure in terms of AUC. In addition, the clinical program performed in human beings reveals that there is no increase of Cₘₐₓ and AUC when increasing the dose from 10 to 20 mg for the crystalline milled drug substance in suspension (cf. AUC_{10 mg}=17.98 μg*h/l vs. AUC_{20 mg}=17.92 μg*h/l). The tablet according to example 2 could be administered up to a much higher dose (e.g. 80 mg and beyond), almost dose-dependently over the entire dosing range.

| | Suspension | Tablet |
|---|---|---|
| AUCₙₒᵣₘ [kg * h/l] | 0.083 | 0.608 |
| C_{max, norm} [kg/l] * 10⁻³ | 1.28 | 8.24 |

It can be demonstrated that this new type of pharmaceutical composition, comprising a solid dispersion of the compound of Formula I, can result in improved bioavailability, and may lead to an overall superior efficacy for the treatment of hyperlipidemic diseases.

1. A pharmaceutical composition comprising a solid dispersion, wherein the solid dispersion comprises implitapide and a pharmaceutically acceptable matrix.

2. The pharmaceutical composition of claim 1, wherein the weight ratio of the implitapide to the pharmaceutically acceptable matrix is about 1:3 to about 1:9.

3. The pharmaceutical composition of claim 2, wherein the weight ratio of the implitapide to the pharmaceutically acceptable matrix is about 1:3 to about 1:4.

4. The pharmaceutical composition of claim 1, wherein a substantial portion of the implitapide is in an amorphous state.

5. The pharmaceutical composition of claim 1, wherein the solubility of the implitapide is increased as compared to the solubility of crystalline implitapide.

6. The pharmaceutical composition of claim 4, wherein the solubility of said implitapide is increased by at least 400-fold.

7. The pharmaceutical composition of claim 1, wherein the pharmaceutically acceptable matrix comprises at least one of: a sugar, cyclodextrin, or a sugar alcohol.

8. The pharmaceutical composition of claim 1, wherein the pharmaceutically acceptable matrix comprises a pharmaceutically acceptable polymer.

9. The pharmaceutical composition of claim 8 wherein the pharmaceutically acceptable polymer is polyvinylpyrrolidone.

10. The pharmaceutical composition of claim 8, wherein the pharmaceutically acceptable polymer is hydroxypropylcellulose.

11. The pharmaceutical composition of claim 2, wherein said pharmaceutical composition is an oral administration composition.

12. A pharmaceutical composition of claim 2, further comprising an additional active ingredient.

13. A tablet comprising a pharmaceutical composition comprising a solid dispersion wherein the solid dispersion comprises implitapide and a pharmaceutically acceptable matrix.

14. The tablet of claim 12, wherein said tablet is an immediate release tablet.

15. A process for manufacturing a pharmaceutical composition comprising a solid dispersion wherein the solid dispersion comprises implitapide and a pharmaceutically acceptable matrix said process comprising:
a) dissolving the implitapide and at least one pharmaceutically acceptable matrix in solvent or a solvent mixture to form a solution;
b) contacting the solution with one or more pharmaceutically acceptable excipients;
c) removing said solvent or solvent mixture to form a granulate; and
d) optionally blending said granulate with one or more further pharmaceutically acceptable excipients to form post-blend granulates.

16. The process of claim 15, further comprising subdividing said post-blend granulates.

17. The process of claim 16, further comprising coating said post-blend granulates with one or more further pharmaceutically acceptable excipients.

18. The process of claim 15, wherein said solvent or solvent mixture comprises acetone.

19. A composition comprising a solid dispersion comprising implitapide, wherein said composition, when administered to a patient, results in a higher exposure, as measured by AUC, of implitapide, as compared to administering to a patient a suspension of substantially crystalline implitapide.

20. The composition of claim 19, wherein the higher exposure is at least about 7-fold higher.

21. The composition of claim 19, wherein the higher exposure is at least about 20-fold higher.

22. The composition of claim 19, wherein said solid dispersion further comprises a pharmaceutically acceptable polymer.

23. A composition comprising implitapide and a pharmaceutically acceptable matrix, wherein the weight ratio of the implitapide to the pharmaceutically acceptable matrix is about 1:3 to about 1:4.

24. A method for treating a hyperlipidemic disorder in a patient in need thereof, comprising administrating a pharmaceutically effective amount of a pharmaceutical composition comprising a solid dispersion, wherein the solid dispersion comprises implitapide and a pharmaceutically acceptable matrix.
